(19) United States
(12) Patent Application Publication
(10) Pub. No.: US 2013/0281462 A1
(43) Pub. Date: Oct. 24, 2013

(54) ARYL DIAMIDINES AND PRODRUGS THEREOF FOR TREATING MYOTONIC DYSTROPHY

(71) Applicant: State of Oregon, acting by and through the State Board of Higher Education, on behalf of the University of Oregon, (US)

(72) Inventors: J. Andrew Berglund, Eugene, OR (US); Micah J. Bodner, Vida, OR (US)

(73) Assignees: University of Oregon, Eugene, OR (US); State of Oregon, acting by and through the State Board of Higher Education, on behalf of the

(21) Appl. No.: 13/866,863

(22) Filed: Apr. 19, 2013

Related U.S. Application Data
(60) Provisional application No. 61/636,398, filed on Apr. 20, 2012.

(51) Int. Cl. C07C 257/18

(52) U.S. Cl. CPC C07C 257/18
USPC 514/255.03; 564/243; 514/636; 549/491; 514/471; 544/392; 548/440; 514/411

(57) ABSTRACT

Disclosed herein are compounds (for example, diamidine derivatives and prodrugs) and methods of use thereof, for example in treating muscular dystrophy (DM) or disease caused by a toxic RNA in a subject. In some embodiments, the methods include administering an effective amount of one of more of the disclosed compounds to a subject to treat or inhibit DM or a disease caused by or associated with toxic RNA, such as DM1, DM2, spinocerebellar ataxia type 8 (SCA8), fragile X tremor ataxia syndrome (FXTAS), or Huntington disease-like 2 (HLD2). In some examples, the methods include selecting a subject for treatment, for example selecting a subject with DM1, DM2, SCA8, FXTAS, or HLD2.

### CROSS REFERENCE TO RELATED APPLICATION

This claims the benefit of U.S. Provisional Application No. 61/636,398, filed Apr. 20, 2012, which is incorporated herein by reference in its entirety.

### ACKNOWLEDGMENT OF GOVERNMENT SUPPORT

This invention was made with government support under Grant No. AR059833 awarded by the National Institutes of Health. The government has certain rights in the invention.

### FIELD

This disclosure relates to diamidine prodrugs and methods for treating toxic RNA diseases, particularly myotonic dystrophy.

### BACKGROUND

Myotonic dystrophy (DM) is the most common form of adult onset muscular dystrophy, affecting 1 in 8000 people. DM is caused by expansion of CTG repeats in type 1 DM (DM1) or CCTG repeats in type 2 DM (DM2). It is believed that production of CUG or CCUG repeats results in a trans-acting RNA that binds and sequesters muscleblind-like protein (MBNL). MBNL proteins regulate alternative RNA splicing and their sequestration in DM leads to mis-splicing events, several of which have been directly linked to symptoms of DM.

No treatment currently exists for either form of DM. Management is generally limited to treatment of the clinical manifestations of the disease, such as assistive devices, pain management, removal of cataracts, and cardiac monitoring. Thus, there is a need for treatments for DM.

### SUMMARY

Disclosed herein are compounds (for example aryl diamidines, and derivatives and prodrugs thereof) and methods of use thereof, for example in treating DM or disease caused by a toxic RNA in a subject.

In some embodiments, the disclosed compounds have the structure:

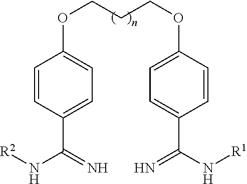

wherein n is 1 to 7, and R¹ and R² are independently selected from H, OR³, C(O)OR⁴, and SR⁵, wherein:

R³ is selected from H, C(O)(CH₂)ₘCH₃, C(O)C(CH₃)₃, (CH₂)ₘOC(O)C(CH₃)₃, SO₂Me, SO₂Tol, and C(O)CH(i-Pr)NH₂, wherein m is 0-10;

R⁴ is selected from (CH₂)ₘCH₃, (CH₂)ₘCX₃, (CH₂)ₘPh, (CH₂)ₘPh-X, (CH₂)ₘPh-Y, and (CH₂)ₘOC(O)C(CH₃)₃, wherein X is independently Cl, F, I, or Br, wherein Y is OR⁶, wherein R⁶ is (CH₂)ₘCH₃, and wherein m is 0-10; and

R⁵ is selected from (CH₂)ₘCH₃, C(CH₃)₃, Ph, and (CH₂)ₘC(O)O(CH₂)ₘCH₃, wherein each m is independently 0-10.

In some embodiments, the methods include administering an effective amount of one of more of the disclosed compounds to a subject to treat or inhibit DM or a disease caused by or associated with toxic RNA, such as DM1, DM2, spinocerebellar ataxia type 8 (SCA8), fragile X tremor ataxia syndrome (FXTAS), or Huntington disease-like 2 (HLD2). In some examples, the methods include selecting a subject for treatment, for example selecting a subject with DM1, DM2, SCA8, FXTAS, or HLD2.

The foregoing and other features of the disclosure will become more apparent from the following detailed description, which proceeds with reference to the accompanying figures.

### BRIEF DESCRIPTION OF THE DRAWINGS

FIG. 1A is a drawing of the structure of pentamidine analogs. The methylene carbon linker is highlighted by parentheses (n=1-7).

FIG. 1B is a series of digital images of electrophoretic mobility shift assays (EMSA) of competition of the indicated compounds with the MBNL1/(CUG)₄ complex.

FIG. 1C is a pair of graphs of IC50 curves for each analog. The left graph shows propamidine, butamidine, and pentamidine on a scale of 0 to 450 μM. The right graph shows pentamidine, hexamidine, heptamidine, octamidine, and nonamidine on a scale from 0 to 50 μM. Error bars are standard deviation for each point.

FIG. 2A is a series of digital images of electrophoresis of reverse transcription-polymerase chain reaction (RT-PCR) reactions showing the effect of pentamidine analogs on splicing of TNNT2 minigene in HeLa cell DM1 model.

FIG. 2B is a series of jitter plots showing TNNT2 minigene splicing in HeLa cell DM1 model. Each point is one experiment and the line represents the average of all experiments for that condition (at least three for each concentration). Gray area denotes range between typical splicing and DM missplicing.

FIG. 2C is a series of digital images of electrophoresis of RT-PCR reactions showing the effect of pentamidine analogs on splicing of INSR minigene in HeLa cell DM1 model.

FIG. 2D is a series of jitter plots showing INSR minigene splicing in HeLa cell DM1 model. Each point is one experiment and the line represents the average of all experiments for that condition (at least three for each concentration). Gray area denotes range between typical splicing and DM missplicing.

FIG. 3 is a graph showing the effect of methylene linker length on EC₅₀ for rescue of missplicing of a TNNT2 or INSR minigene.

FIG. 4A is a graph showing percent inclusion of Clcn1 exon 7a in vastus muscle of HSA^{LR} mice treated with the indicated amount of heptamidine (top) and a digital image of RT-PCR data (in duplicate) for each treatment condition. Gray area in graph denotes range between typical splicing and DM1 missplicing. Complete rescue occurred with 20 mg/kg heptamidine for 7 days. After treatment, withdrawal mice (WD) were maintained for 10 days with no additional treatment. These mice showed a complete return to disease state splicing levels.

FIG. 4B is a graph showing percent inclusion of Atp2a1 exon 22 in vastus muscle of HSA^{LR} mice treated with the indicated amount of heptamidine (top) and a digital image of RT-PCR data (in duplicate) for each treatment condition. Gray area in graph denotes range between typical splicing and DM1 missplicing. Partial rescue of missplicing was achieved, with about 50% rescue with 30 mg/kg heptamidine treatment. After treatment, withdrawal mice (WD) were maintained for 10 days with no additional treatment. These mice showed a complete return to disease state splicing levels.

FIG. 5 is a graph showing myotonia rescue in HSALR mice treated with 0, 20, or 30 mg/kg heptamidine for 7 days. Untreated mice showed myotonic discharge with nearly all electrode insertions (grade 3), whereas the treated mice had occasional myotonic discharges with less than 50% of insertions (grade 1) or no myotonia (grade 0).

FIG. 6 is a bar graph showing the effect of pentamidine derivatives 128 and 136 on INSR missplicing. The gray area denotes the range between typical splicing and DM1 missplicing.

### SEQUENCE LISTING

Any nucleic acid and amino acid sequences listed herein or in the accompanying sequence listing are shown using standard letter abbreviations for nucleotide bases and amino acids, as defined in 37 C.F.R. §1.822. In at least some cases, only one strand of each nucleic acid sequence is shown, but the complementary strand is understood as included by any reference to the displayed strand.

The Sequence Listing is submitted as an ASCII text file in the form of the file named Sequence_Listing.txt, which was created on Apr. 18, 2013, and is 1948 bytes, which is incorporated by reference herein.

SEQ ID NO: 1 is the nucleic acid sequence of a (CUG)₄ RNA molecule.

SEQ ID NOs: 2-4 are the nucleic acid sequences of TNNT2 RT-PCR primers.

SEQ ID NOs: 5-7 are the nucleic acid sequences of INSR RT-PCR primers.

### DETAILED DESCRIPTION

Diamidine analogs with varying methylene chain length (particularly pentamidine and heptamidine) have been identified as promising lead compounds for the treatment of DM and other diseases caused by or associated with toxic RNA (disclosed herein and in U.S. Pat. App. Publ. No. 2010/0323993, incorporated herein by reference). It is desirable to identify derivatives or prodrugs of these compounds with improved properties, such as increased oral bioavailability, decreased toxicity, and increased efficacy.

Disclosed herein are diamidine derivatives (such as aryl diamidines) and prodrugs thereof which retain activity of the originally identified compounds (for example, ability to rescue missplicing in in vitro and/or in vivo DM models), while exhibiting reduced cellular toxicity. In some examples, the disclosed compounds are therapeutically effective at lower doses than the lead compounds. Such compounds could treat, inhibit, or even prevent one or more symptoms of DM or other toxic RNA disease with minimal toxic side effects.

### I. ABBREVIATIONS

ATP2a1: ATPase, Ca++ transporting, cardiac muscle, fast twitch 1

Clcn1: chloride channel, voltage-sensitive 1

DM: myotonic dystrophy

DM1: myotonic dystrophy type I

DM2: myotonic dystrophy type II

DMPK: dystrophia myotonica-protein kinase gene or protein

EMSA: electrophoretic mobility shift assay

FXTAS: fragile X tremor ataxia syndrome

HDL2: Huntington disease-like 2

INSR: insulin receptor

MBNL: muscleblind-like gene or protein

SCAB: spinocerebellar ataxia type 8

TNNT2: troponin T type 2 (cardiac)

UTR: untranslated region

ZNF9: zinc finger 9 gene or protein

### II. TERMS

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. Hence “comprising A or B” means including A, or B, or A and B. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of the present disclosure, suitable methods and materials are described below.

All publications, patent applications, patents, and other references mentioned herein are incorporated by reference in their entirety. All GenBank Accession numbers mentioned herein are incorporated by reference in their entirety as present in GenBank on Jan. 6, 2012. In case of conflict, the present specification, including explanations of terms, will control. In addition, the materials, methods, and examples are illustrative only and not intended to be limiting.

In order to facilitate review of the various embodiments of the disclosure, the following explanations of specific terms are provided:

Diamidine: Any of a group of compounds containing two of the groups —C(═NH)NH₂ (amidine) or derivatives thereof. The amidine groups are connected by a linker, which may be of varying size or length. In one example, the diamidine is pentamidine, which contains two amidine groups connected by a five carbon chain. Other diamidines include propamidine, butamidine, hexamidine, and heptamidine. Diamidines also include derivatives of diamidines (such as aryl diamidines), in which at least one atom is added, removed, or replaced by at least one other atom.

Myotonic dystrophy: A complex neuromuscular disorder, characterized by myotonia with muscle weakness and wasting, cataracts, cardiac conduction defects, insulin resistance, neuropsychiatric impairment, and other developmental or degenerative manifestations. DM1 is caused by a CTG repeat expansion within the 3′ untranslated region (UTR) of the DMPK gene, while DM2 is caused by a CCTG repeat expansion in intron 1 of the ZNF9 gene. Both DM1 and DM2 exhibit similar symptoms.

Nucleotide repeat expansion: A type of mutation in which a set of repeated sequences replicates inaccurately to increase the number of repeats above that normally present in a nucleic acid sequence. Nucleotide repeat expansions include increases in number of trinucleotide, tetranucleotide, and pentanucleotide repeat sequences. The expansions may be present in both DNA and RNA and may be translated or non-translated. In some examples, a nucleotide repeat expansion in a non-coding RNA molecule produces a toxic RNA, which may result in cellular damage or disease, potentially through a gain-of-function mechanism.

Myotonic dystrophy is a disease which is caused by a nucleotide repeat expansion. For example, a CTG nucleotide repeat expansion is present in either the 3′UTR or the last exon of the DMPK gene (DNA) in DM1. Upon transcription of the DMPK gene, a CUG nucleotide repeat expansion is present in the resulting RNA or mRNA. Similarly, a CCTG repeat expansion is present in intron 1 of the ZNF9 gene (DNA) in DM2. Upon transcription, a CCUG nucleotide repeat expansion is present in the resulting ZNF9 RNA transcript, although the expansion is not present in appropriately spliced messenger RNA, where the intron has been removed.

Many nucleotide repeat expansions are associated with disease. For example, trinucleotide repeat expansions in different genes are associated with diseases such as myotonic dystrophy type I (CTG repeat expansion), Fragile X syndrome (CGG repeat expansion), Friedrich ataxia (GAA repeat expansion), Huntington disease (CAG repeat expansion), and several spinocerebellar ataxias (CAG repeat expansions). In other examples of nucleotide repeat expansions associated with disease, a tetranucleotide repeat expansion is associated with myotonic dystrophy type II (CCTG repeat expansion) and a pentanucleotide repeat expansion is associated with spinocerebellar ataxia type 10 (ATTCT repeat expansion).

Preventing, treating or ameliorating: “Preventing” a disease or condition refers to inhibiting the full development of a disease or condition. “Treating” refers to a therapeutic intervention that ameliorates a sign or symptom of a disease or pathological condition after it has begun to develop. “Ameliorating” refers to the reduction in the number or severity of signs or symptoms of a disease or condition. In one embodiment, the disease or condition is myotonic dystrophy.

Prodrug: Any covalently bonded carriers that release a disclosed compound or a parent thereof in vivo when the prodrug is administered to a subject. Prodrugs often have enhanced properties relative to the active agent pharmaceutical, such as solubility and bioavailability. Prodrugs of the disclosed compounds typically are prepared by modifying one or more functional groups present in the compound in such a way that the modifications are cleaved, either in routine manipulation or in vivo, to yield the parent compound. Conventional procedures for the selection and preparation of suitable prodrug derivatives are described, for example, in Design of Prodrugs, ed. H. Bundgaard, Elsevier, 1985. In one example, a prodrug is pafuramidine.

Subject: Living multi-cellular vertebrate organisms, a category that includes human and non-human mammals (such as laboratory or veterinary subjects).

Therapeutically effective amount: A dose sufficient to prevent advancement, delay progression, or to cause regression of the disease, or which is capable of reducing symptoms caused by the disease, such as myotonic dystrophy.

Toxic RNA: RNA which contains a non-coding nucleotide repeat expansion and which results in cellular damage or disease. In some instances, toxic RNA may accumulate in the nucleus, sequester binding proteins, and result in abnormal splicing for some pre-mRNAs (Osborne & Thornton, Hum. Mol. Genet. 15:R162-R169, 2006). In a particular example, a CUG trinucleotide repeat expansion in the 3′ UTR of the DMPK gene results in production of a toxic RNA which leads to DM1. In another example, a CCUG tetranucleotide repeat expansion in intron 1 of the ZNF9 gene produces a toxic RNA which leads to DM2. Other diseases which may be caused by production of toxic RNA include Fragile X tremor ataxia syndrome (FXTAS), spinocerebellar ataxia type 8 (SCA8), and Huntington disease-like 2 (HDL2).

### III. ARYL DIAMIDINE AND PRODRUG COMPOUNDS

Disclosed herein are aryl diamidines or derivatives or prodrugs of diamidines which may be used for treating toxic RNA diseases (for example, myotonic dystrophy). In some embodiments, the disclosed compounds include a modification of one or both of the amidine groups of the diamidine.

In some examples, the disclosed compounds include the following structure:

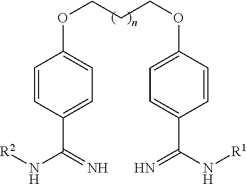

wherein n is 1 to 7, and R¹ and R² are independently selected from H, OR³, C(O)OR⁴, and SR⁵, wherein:

R³ is selected from H, C(O)(CH₂)ₘCH₃, C(O)C(CH₃)₃, (CH₂)ₘOC(O)C(CH₃)₃, SO₂Me, SO₂Tol, and C(O)CH(i-Pr)NH₂, wherein m is 0-10;

R⁴ is selected from (CH₂)ₘCH₃, (CH₂)ₘCX₃, (CH₂)ₘPh, (CH₂)ₘPh-X, (CH₂)ₘPh-Y, and (CH₂)ₘOC(O)C(CH₃)₃, wherein X is independently Cl, F, I, or Br, wherein Y is OR⁶, wherein R⁶ is (CH₂)ₘCH₃, and wherein m is 0-10; and

R⁵ is selected from (CH₂)ₘCH₃, C(CH₃)₃, Ph, and (CH₂)ₘC(O)O(CH₂)ₘCH₃, wherein each m is independently 0-10.

In some examples, the compounds have the structure:

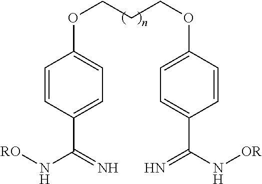

wherein n is 1 to 7 and R is selected from H, C(O)(CH₂)ₘCH₃, C(O)C(CH₃)₃, (CH₂)ₘOC(O)C(CH₃)₃, SO₂Me, SO₂Tol, and C(O)CH(i-Pr)NH₂, wherein m is 0-10. In one particular example, R is C(O)CH₃.

In other examples, the compounds have the structure:

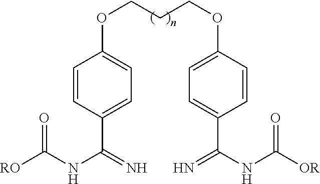

wherein n is 1 to 7 and R is selected from (CH₂)ₘCH₃, (CH₂)ₘCX₃, (CH₂)ₘPh, (CH₂)ₘPh-X, (CH₂)ₘPh-Y, and (CH₂)ₘOC(O)C(CH₃)₃, wherein X is independently Cl, F, I, or Br, wherein Y is OR⁶, wherein R⁶ is (CH₂)ₘCH₃, and wherein m is 0-10. In a specific example, R is Ph(O)CH₃.

In further examples, the compounds have the structures:

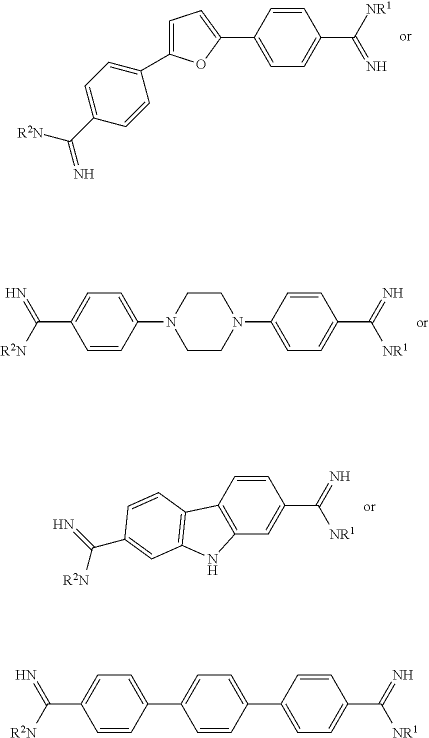

for each, wherein R¹ and R² are independently selected from H, OR³, C(O)OR⁴, and SR⁵, wherein:

R³ is selected from H, C(O)(CH₂)ₘCH₃, C(O)C(CH₃)₃, (CH₂)ₘOC(O)C(CH₃)₃, SO₂Me, SO₂Tol, and C(O)CH(i-Pr)NH₂, wherein m is 0-10;

R⁴ is selected from (CH₂)ₘCH₃, (CH₂)ₘCX₃, (CH₂)ₘPh, (CH₂)ₘPh-X, (CH₂)ₘPh-Y, and (CH₂)ₘOC(O)C(CH₃)₃, wherein X is independently Cl, F, I, or Br, wherein Y is OR⁶, wherein R⁶ is (CH₂)ₘCH₃, and wherein m is 0-10; and

R⁵ is selected from (CH₂)ₘCH₃, C(CH₃)₃, Ph, and (CH₂)ₘC(O)O(CH₂)ₘCH₃, wherein each m is independently 0-10.

### IV. METHODS OF TREATING DM OR OTHER TOXIC RNA DISEASE

The compounds described herein (including, but not limited to, those described in Section III, above) may be used to decrease or prevent DM phenotypes in a human or animal subject with DM1 or DM2 (for example, to treat, inhibit, or in some instances prevent DM in a subject). The compounds may also be used to decrease or prevent phenotypes of a disease caused by or associated with toxic RNA in a human or animal subject, such as a subject with DM, SCA8, FXTAS, or HLD2 (for example to treat, inhibit, or in some instances prevent a toxic RNA disease in a subject). In some examples, the methods include selecting a subject with DM (for example, DM1 or DM2) for treatment with an aryl diamidine or other diamidine derivative or prodrug, such as those disclosed herein. In other examples, the methods include selecting a subject with a disease caused by a toxic RNA (for example, DM, SCAB, FXTAS, or HLD2) for treatment with an aryl diamidine or derivative or prodrug thereof, such as those disclosed herein.

Pharmaceutical compositions that include an aryl diamidine, derivative, or prodrug thereof (or a combination of two or more thereof, such as 1, 2, 3, or 4) can be formulated with an appropriate pharmaceutically acceptable carrier, depending upon the particular mode of administration chosen. The pharmaceutically acceptable carriers and excipients useful in this disclosure are conventional. See, e.g., Remington: The Science and Practice of Pharmacy, The University of the Sciences in Philadelphia, Editor, Lippincott, Williams, & Wilkins, Philadelphia, Pa., 21^{at} Edition (2005). For instance, parenteral formulations usually comprise injectable fluids that are pharmaceutically and physiologically acceptable fluid vehicles such as water, physiological saline, other balanced salt solutions, aqueous dextrose, glycerol or the like. For solid compositions (e.g., powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, pH buffering agents, or the like, for example sodium acetate or sorbitan monolaurate.

In some examples, the aryl diamidine or diamidine derivative or prodrug (such as those disclosed herein) includes a pharmaceutically acceptable salt of such compounds. Pharmaceutically acceptable salts of the presently disclosed compounds include those formed from cations such as sodium, potassium, aluminum, calcium, lithium, magnesium, zinc, and from bases such as ammonia, ethylenediamine, N-methyl-glutamine, lysine, arginine, ornithine, choline, N,N′-dibenzylethylenediamine, chloroprocaine, diethanolamine, procaine, N-benzylphenethylamine, diethylamine, piperazine, tris(hydroxymethyl)aminomethane, and tetramethylammonium hydroxide. These salts may be prepared by standard procedures, for example by reacting the free acid with a suitable organic or inorganic base. Any chemical compound recited in this specification may alternatively be administered as a pharmaceutically acceptable salt thereof. Pharmaceutically acceptable salts are also inclusive of the free acid, base, and zwitterionic forms. Description of suitable pharmaceutically acceptable salts can be found in Handbook of Pharmaceutical Salts, Properties, Selection and Use, Wiley VCH (2002).

The compounds of this disclosure can be administered to humans or other animals on whose tissues they are effective in various manners such as orally, intravenously, intramuscularly, intraperitoneally, intranasally, intradermally, intrathecally, subcutaneously, via inhalation or via suppository. In one non-limiting example, the compound is administered orally. In another non-limiting example, the compound is administered intraperitoneally. The particular mode of administration and the dosage regimen will be selected by the attending clinician, taking into account the particulars of the case (e.g. the subject, the disease, and the disease state involved). Treatment can involve daily or multi-daily doses of compound(s) over a period of a few days to months, or even years. The compound(s) may be administered about every 6 hours, about every 12 hours, about every 24 hours (daily), about every 48 hours, about every 72 hours, or about weekly. In one non-limiting example, the compound(s) are administered orally once per day. Treatment with repeated doses may continue for a period of time, for example for about 1 week to 12 months, such as about 1 week to about 6 months, or about 2 weeks to about 3 months, or about 1 to 2 months.

One of skill in the art can identify appropriate doses for the diamidine derivative or prodrug of use in the disclosed methods. The amount administered will be dependent on factors such as the subject being treated, the type and severity of the condition (for example, DM1 or DM2), and the mode of administration. A pharmaceutical composition that includes one or more diamidine derivatives or prodrugs can be formulated in unit dosage form, suitable for individual administration of precise dosages. In one specific, non-limiting example, a unit dosage contains from about 1 mg to about 5 g of an aryl diamidine or diamidine derivative or prodrug (such as about 100 mg to about 2.5 g, about 250 mg to about 1 g, about 500 mg to about 750 mg, or about 100 mg to about 500 mg). In some examples, a unit dosage contains about 1 mg, 10 mg, 25 mg, 100 mg, 250 mg, 500 mg, 750 mg, 1 g, 1.5 g, 2 g, 2.5 g, 3 g, 4 g, or 5 g of the aryl diamidine, derivative, or prodrug. The amount of active compound(s) administered will be dependent on the subject being treated, the severity of the affliction, and the manner of administration, and is best left to the judgment of the prescribing clinician. Within these bounds, the formulation to be administered will contain a quantity of the active component(s) in amounts effective to achieve the desired effect in the subject being treated.

In some examples, a therapeutically effective amount of an aryl diamidine or diamidine derivative or prodrug is about 0.1 mg/kg to about 100 mg/kg (for example, about 1 mg/kg to about 50 mg/kg, about 10 mg/kg to about 25 mg/kg, or about 0.5 mg/kg to about 5 mg/kg). In a specific example, a therapeutically effective amount of an aryl diamidine or diamidine derivative or prodrug is about 0.5 mg/kg to about 30 mg/kg, such as about 0.5 mg/kg, 1 mg/kg, 2 mg/kg, 3 mg/kg, 4 mg/kg, 5 mg/kg, 10 mg/kg, 15 mg/kg, 20 mg/kg, 25 mg/kg, or 30 mg/kg. In some examples, a therapeutically effective amount of an aryl diamidine or diamidine derivative or prodrug is about 0.5 mg/kg/day to about 100 mg/kg/day (for example, about 1 mg/kg/day to about 50 mg/kg/day, about 10 mg/kg/day to about 30 mg/kg/day, or about 0.5 mg/kg/day to about 10 mg/kg/day). One of skill in the art can extrapolate from an animal dose (such as a rat or mouse) to an appropriate human dose (see, e.g., Reagan-Shaw et al., FASEB J. 22:659-661, 2008).

Methods of assessing DM phenotypes are well known to those of skill in the art. DM phenotypes in affected individuals include muscle weakness (which may lead to foot drop and gait disturbance, as well as difficulty in performing tasks requiring hand dexterity), myotonia (sustained muscle contraction), which often manifests as the inability to quickly release a hand grip (grip myotonia) and which can be demonstrated by tapping a muscle with a reflex hammer (percussion myotonia), and myotonic discharges observed by electromyography recording. Pathologic features may be observed by muscle biopsy, including rows of internal nuclei, ring fibers, sarcoplasmic masses, type I fiber atrophy, and increased number of intrafusal muscle fibers.

Changes in DM phenotypes may be monitored in DM subjects following administration of compounds, such as diamidine derivatives or prodrugs. DM phenotypes may be compared to DM subjects who have not received the compounds or comparison may be made to the subject's phenotype prior to administration of the compound in order to assess effectiveness of the compound for treatment of DM.

With the teaching herein that diamidine derivatives and prodrugs (such as those disclosed herein) are useful for treating myotonic dystrophy, it will now be understood that these therapies also have broader application in other diseases and conditions caused (or influenced) by toxic RNA, particularly other conditions or diseases that involve nucleotide repeat expansion toxicity. For a review of toxic RNA (or RNA-dominant) diseases, see Osborne and Thornton, Hum. Mol. Genet. 15:R162-R169, 2006. Toxic RNA diseases include those with a mutation in a non-coding region which produce RNAs that have a deleterious gain-of-function effect. In some examples, the mutation is a nucleotide repeat expansion (such as a trinucleotide or tetranucleotide repeat expansion) which is located in an intron or an untranslated region. Without being bound by theory, toxic RNAs may cause pathology by exerting a trans-effect on the alternative splicing of many pre-mRNAs; thus, rather than leading to the production of a mutant protein, they lead to expression of inappropriate splice products in a particular tissue or at a particular developmental stage. In a particular example, CUG repeat expansions, such as in DM1, lead to sequestration of muscleblind-like proteins in ribonuclear foci and depletion in other parts of the nucleoplasm. However, not all toxic RNA diseases are expected to be caused by an identical mechanism.

Myotonic dystrophy is the prototypical toxic RNA disease. Spinocerebellar ataxia type 8 (SCA8) and fragile X tremor ataxia syndrome (FXTAS) are representative additional diseases. SCA8 is caused by CUG repeat expansions in a non-coding RNA (ATXN8OS/SCA8), while in FXTAS there is an expansion of about 70-120 CGG repeats in the 5′ UTR of the FMR1 gene. Another toxic RNA disease is Huntington disease-like 2 (HDL2), which is caused by a CUG repeat expansion in an intron or the 3′ UTR of the junctophilin 3 gene.

It will be understood that the methods and compositions described herein for treating DM, comprising administering a compound that binds a nucleotide repeat expansion (such as a DNA or RNA nucleotide repeat expansion), are applicable to methods of treating toxic RNA diseases, such as those described above. The methods for assessing the effectiveness of test compounds for treating such diseases in cells, appropriate animal models, or affected subjects are known to one of skill in the art. For example, animal models of FXTAS (Jin et al., Neuron 39:739-747, 2003; Brouwer et al., Exp. Cell Res. 313:244-253, 2007) and SCA8 (Mutsuddi et al., Curr. Biol. 14:302-308, 2004; Moseley et al., Nature Genet. 38:758-769, 2006) are known to those in the art.

The present disclosure is illustrated by the following non-limiting Examples.

### EXAMPLES

### Example 1

### Effect of Methylene Linker Length on Mis-Splicing In Vitro

### Methods

MBNL Purification:

The MBNL1 medium construct (1-260) was purified as previously described (Warf et al., Proc. Natl. Acad. Sci. USA 106:18551-18556, 2009) with the following changes: after purification, the protein was dialyzed into 500 mM NaCl, 20 mM Tris pH 7.5, 50% (v/v) glycerol, and 5 mM β-mercaptoethanol. Aliquots were stored at −80° C. until just before use.

RNA Labeling:

The (CUG)₄ RNA construct used in the in vitro EMSA (5′-GCUGCUGUUCGCUGCUG; SEQ ID NO: 1) was ordered from IDT (Coralville, Iowa) and 5′ end labeled using [γ³²P]ATP. After phosphorylation the RNA was purified using a spin column containing Bio-Gel P2 Gel (Bio-Rad, Hercules, Calif.), brought to final stock concentration of 50 nM using low TE, and stored at −20° C. until use.

Synthesis of Pentamidine Analogues:

Pentamidine isethionate was purchased from Sigma-Aldrich (St. Louis, Mo.) and the HCl salt was accessed by recrystallization from hot 10% aqueous HCl. Propamidine, butamidine, and hexamidine were synthesized as described by Tidwell et al. (J. Med. Chem. 33:1252-1257, 1990). Heptamidine, octamidine and nonamidine were prepared in an analogous fashion.

Propamidine: White powder. ¹H NMR (600 MHz, (CD3)₂SO) δ 2.34 (pent, J=6.0 Hz, 2H), 4.27 (t, J=6.0 Hz, 4H), 7.18 (d, J=9.0 Hz, 4H), 7.86 (d, J=9.0 Hz, 4H), 9.08 (br s, 4H), 9.28 (br s, 4H); ¹³C NMR (150 MHz, (CD3)₂SO) δ 28.22, 64.80, 114.80, 119.52, 130.22, 162.81, 164.67.

Butamidine: White powder. ¹H NMR (600 MHz, (CD3)₂SO) δ 1.91 (pent, J=6.0 Hz, 4H), 4.17 (t, J=6.0 Hz, 4H), 7.15 (d, J=9.0 Hz, 4H), 7.88 (d, J=9.0 Hz, 4H), 9.12 (br s, 4H), 9.31 (br s, 4H); ¹³C NMR (150 MHz, (CD3)₂SO) δ 25.12, 67.73, 114.77, 119.31, 130.20, 162.99, 164.70.

Pentamidine: White powder. ¹H NMR (600 MHz, (CD3)₂SO) δ 1.58 (pent, J=7.2 Hz, 2H), 1.80 (pent, J=6.0, 7.2 Hz, 4H), 4.15 (t, J=6.0 Hz, 4H), 7.14 (d, J=9.0 Hz, 4H), 7.86 (d, J=9.0 Hz, 4H), 9.11 (br s, 4H), 9.29 (br s, 4H); ¹³C NMR (150 MHz, (CD3)₂SO) δ 22.50, 28.60, 68.46, 115.19, 119.69, 130.63, 163.49, 165.14.

Hexamidine: White powder. ¹H NMR (600 MHz, (CD3)₂SO) δ 1.48 (m, 4H), 1.76 (pent, J=6.0 Hz, 4H), 4.09 (t, J=6.0, 4H), 7.13 (d, J=9.0 Hz, 4H), 7.87 (d, J=9.0 Hz, 4H), 9.11 (br s, 4H), 9.30 (br s, 4H); ¹³C NMR (150 MHz, (CD3)₂SO) δ 25.60, 28.83, 68.47, 115.18, 119.66, 130.63, 163.49, 165.14.

Heptamidine: White powder. ¹H NMR (600 MHz, (CD3)₂SO) δ 1.43 (m, 6H), 1.74 (m, 4H), 4.08 (t, J=6.6 Hz, 4H), 7.13 (d, J=8.4 Hz, 4H), 7.88 (d, J=8.4 Hz, 4H), 9.14 (br s, 4H), 9.31 (br s, 4H); ¹³C NMR (150 MHz, (CD3)₂SO) δ 25.79, 28.81, 28.87, 68.50, 115.17, 119.62, 130.63, 163.50, 165.15.

Octamidine: White powder. ¹H NMR (600 MHz, (CD3)₂SO) δ 1.35 (m, 4H), 1.42 (m, 4H), 1.73 (m, 4H), 4.07 (t, J=6.6 Hz, 4H), 7.13 (d, J=8.4 Hz, 4H), 7.87 (d, J=8.4 Hz, 4H), 9.13 (br s, 4H), 9.31 (br s, 4H); ¹³C NMR (150 MHz, (CD3)₂SO) δ 25.79, 28.87, 29.10, 68.52, 115.16, 119.62, 130.62, 163.51, 165.15.

Nonamidine: White powder. ¹H NMR (600 MHz, (CD3)₂SO) δ 1.32 (m, 6H), 1.40 (m, 4H), 1.73 (m, Hz, 4H), 4.07 (t, J=6.6 Hz, 4H), 7.13 (d, J=9.0 Hz, 4H), 7.86 (d, J=9.0 Hz, 4H), 9.09 (br s, 4H), 9.28 (br s, 4H); ¹³C NMR (150 MHz, (CD3)₂SO) δ 25.83, 28.88, 29.11, 29.36, 68.53, 115.17, 119.65, 130.62, 163.51, 165.14.

Gel Shift Assay:

The 5′ end labeled (CUG)₄ RNA was heated at 95° C. for 3 minutes in 250 mM NaCl, 25 mM MgCl₂, and 75 mM Tris pH 7.5 by heating at 95° C. for 3 minutes. The reaction was cooled on ice for 5 minutes, BSA and heparin were added, and the reaction cooled another 5 minutes. MBNL1 was then added to the RNA and incubated at room temperature (RT) for 10 minutes. Amidine derivative was added, followed by another 5 minute RT incubation, followed by the addition of bromophenol blue. The final reaction volume of 10 μL was incubated at RT for 25 minutes. Final reaction conditions were 0.5 nM (CUG)₄, 175 mM NaCl, 20 mM Tris pH 7.5, 5 mM MgCl₂, 2 mg/mL BSA, 0.1 mg/mL heparin, 1.25 mM β-mercaptoethanol, 1.25% (v/v) glycerol, 250 μM bromophenol blue, and 250 nM MBNL. Bromophenol blue was added at 250 μM since the activity of pentamidine is dependent upon bromophenol blue in vitro. Then 3 μL of the reaction was run on a 5% (w/v) 80:1 pre-chilled polyacrylamide gel with 0.5×TB. Gels were run at 4° C. at 170V for 35 minutes, dried and autoradiographed. IC₅₀ values were calculated as previously described (Warf et al., Proc. Natl. Acad. Sci. USA 106:18551-18556, 2009). In brief, gels were quantified using ImageQuant™ software (Molecular Dynamics/GE Healthcare, Piscataway, N.J.). The percent of RNA bound was determined by taking the ratio of the RNA:protein complex (including any well shifting) to the total RNA, per lane. IC₅₀ values were determined with KaleidaGraph (Synergy, Reading, Pa.) software using the equation:

$$Y = \frac{{m}_{3}}{1 + {( \frac{{m}_{0}}{{m}_{1}} )}^{{m}_{2}}}$$

where m₀=small molecule concentration, m₁=IC₅₀, m₂=Hill coefficient, and m₃=fraction of MBNL1 bound without small molecule present. Errors were determined by calculating the standard deviation of triplicate data.

Splicing Analysis in Cell Culture:

Splicing was performed as described previously (Warf et al., Proc. Natl. Acad. Sci. USA 106:18551-18556, 2009) with the following changes. Approximately 2×10⁵ cells were plated in 6 well plates and transfected 24-36 hours later. After transfection, cells were incubated in Opti-MEM™ medium (Life Technologies, Carlsbad, Calif.) for 6 hours and then washed with 1×PBS and placed in DMEM with GlutaMAX™ (Life Technologies) supplemented with 10% FBS (Life Technologies) along with pentamidine analogues. Cells were harvested after 16-18 hours and RNA was isolated immediately using an RNeasy® kit (Qiagen, Valencia, Calif.). After DNase treatment, RNA was reverse transcribed with SuperScript II® and a plasmid specific reverse primer. This cDNA was then subjected to PCR (22 rounds for INSR or 24 rounds for TNNT2). Resulting PCR products were then run on a 6% 19:1 native polyacrylamide gel containing 0.5×TB at 300 V for 90 minutes. The gel was then stained with 1×SYBR I dye (Applied Biosystems, Foster City, Calif.) in 0.5×TB for 15 minutes. Quantification of bands was performed using the Alpha Imager HP software from Alpha Innotech.

The following primers were used. For the TNNT2 minigene, the RT primer was 5′-AGCATTTAGGTGACACTATAGAATAGGG (SEQ ID NO: 2). The forward primer for PCR was 5′-GTTCACAACCATCTAAAGCAAGATG (SEQ ID NO: 3) and the reverse primer was 5′-GTTGCATGGCTGGTGCAGG (SEQ ID NO: 4). For the INSR minigene, the RT primer was 5′-GCTGCAATAAACAAGTTCTGC (SEQ ID NO: 5). The forward primer for PCR was 5=CGAATTCGAATGCTGCTCCTGTCCAAAGACAG (SEQ ID NO: 6), and the reverse primer was 5′-TCGTGGGCACGCTGGTCGAG (SEQ ID NO: 7).

### Results

In Vitro Efficacy Increases with Linker Length:

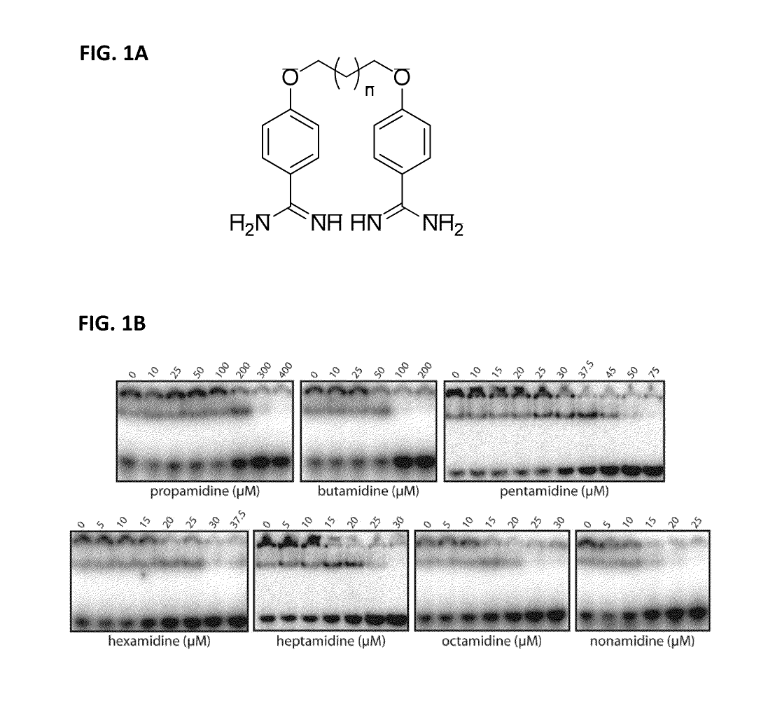

The length of pentamidine's methylene linker was modified. The most polar analogue was propamidine, with a three carbon linker, and the least polar was nonamidine, with a linker of nine carbons (FIG. 1A). The activity of these compounds were tested using the competitive EMSA in which the amount of MBNL1 and (CUG)₄ (a small hairpin RNA composed of two CUG repeats, a UUCG cap, and two more CUG repeats) were held constant. As molecules are added, those effective at disrupting the MBNL1/(CUG)₄ complex resulted in an increase in free RNA. We found that propamidine had the lowest activity, with an IC₅₀ of 250±10 μM, followed by butamidine (IC₅₀=85±17 μM), both of which were worse competitors than pentamidine (IC₅₀=39±2 μM) (FIGS. 1B and C; Table 1). Analogues with longer linkers showed an improvement over pentamidine. Hexamidine disrupted the MBNL1/(CUG)₄ complex with an IC₅₀ of 26±2 μM. Heptamidine and octamidine were comparable with IC₅₀ values of 20±2 μM and 20±1 μM, respectively. Nonamidine was the most effective in our competitive EMSA with an IC₅₀ of 14±1 μM (FIGS. 1B and C; Table 1). These results showed that analogues containing longer oligomethylene linkers are more effective at disrupting the MBNL1/(CUG)₄ complex.

TABLE 1
IC₅₀ of diamidine analogs

| Compound | Linker length | IC₅₀ (μM) |
|---|---|---|
| Propamidine | 3 | 250 ± 10 |
| Butamidine | 4 | 85 ± 17 |
| Pentamidine | 5 | 39 ± 2 |
| Hexamidine | 6 | 26 ± 2 |
| Heptamidine | 7 | 20 ± 2 |
| Octamidine | 8 | 20 ± 1 |
| Nonamidine | 9 | 14 ± 1 |

Pentamidine Analogues Rescue Two Mis-Spliced Minigenes in Tissue Culture:

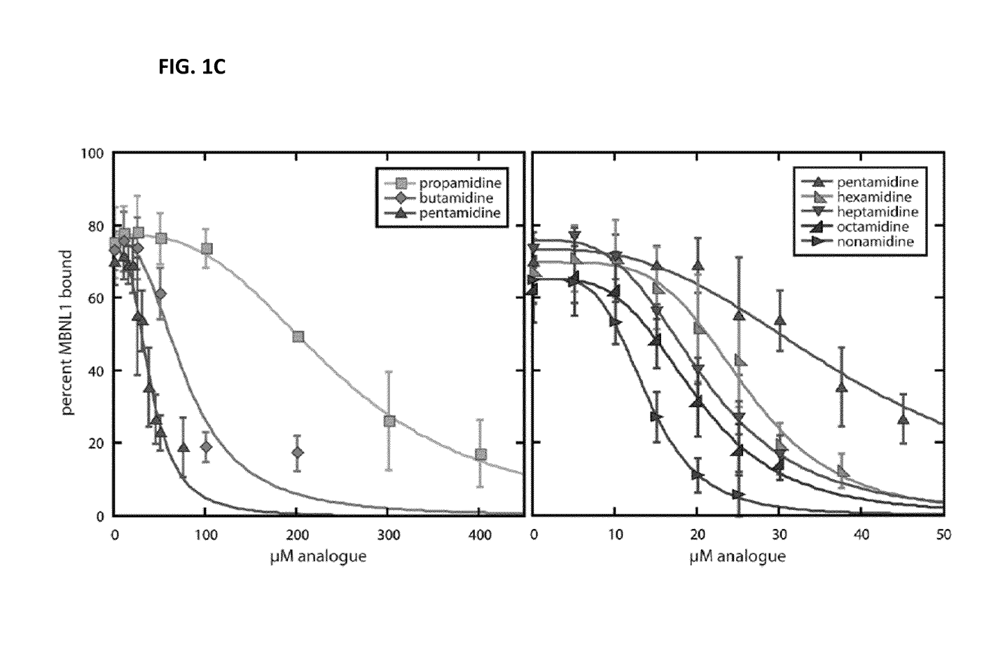

To determine if the trend observed in the competitive EMSA held true in a cell-based system, each analogue was tested for the ability to rescue splicing of transiently transfected minigenes in a HeLa cell model of DM1. Two different minigenes containing exons that are mis spliced in DM were tested: TNNT2 (also known as cTNT), containing the alternatively spliced exon 5, and INSR, containing the alternatively spliced exon 11. MBNL proteins facilitate exclusion (negative regulation) of exon 5 of TNNT2: when MBNL proteins are present, exon 5 is excluded. The wild type level of exon 5 inclusion when the TNNT2 minigene was expressed was 64±2%. This increased to 82±3% when HeLa cells also expressed a DMPK plasmid containing 960 CUG repeats (FIG. 2A). Presumably, this change in TNNT2 exon 5 inclusion is due to the sequestration of endogenous MBNL proteins to the CUG repeats. Propamidine did not have the desired effect on TNNT2 mis-splicing at concentrations up to 130 μM (higher concentrations were toxic), and, if anything, caused slightly higher levels of exon 5 inclusion. Also unable to rescue splicing defects before causing significant cell death (at 4 μM) was nonamidine. The remaining linker analogues rescued TNNT2 mis-splicing to varying degrees. The concentration needed to observe 50% rescue (EC50) for butamidine was 23±5 μM, which was similar to pentamidine (EC₅₀=20±4 μM). At higher concentrations, butamidine and pentamidine lowered exon 5 inclusion levels below that of wild type. Hexamidine and heptamidine showed improvements over pentamidine with EC₅₀ values of 12±3 μM and 15±6 μM, respectively. Finally, octamidine showed a slight rescue of TNNT2 mis-splicing; however, because of toxicity issues, it was not possible treat the cells with high enough concentrations to obtain an accurate EC₅₀ value (FIG. 2B). Thus, hexamidine was able to rescue TNNT2 missplicing at the lowest dose while demonstrating low toxicity.

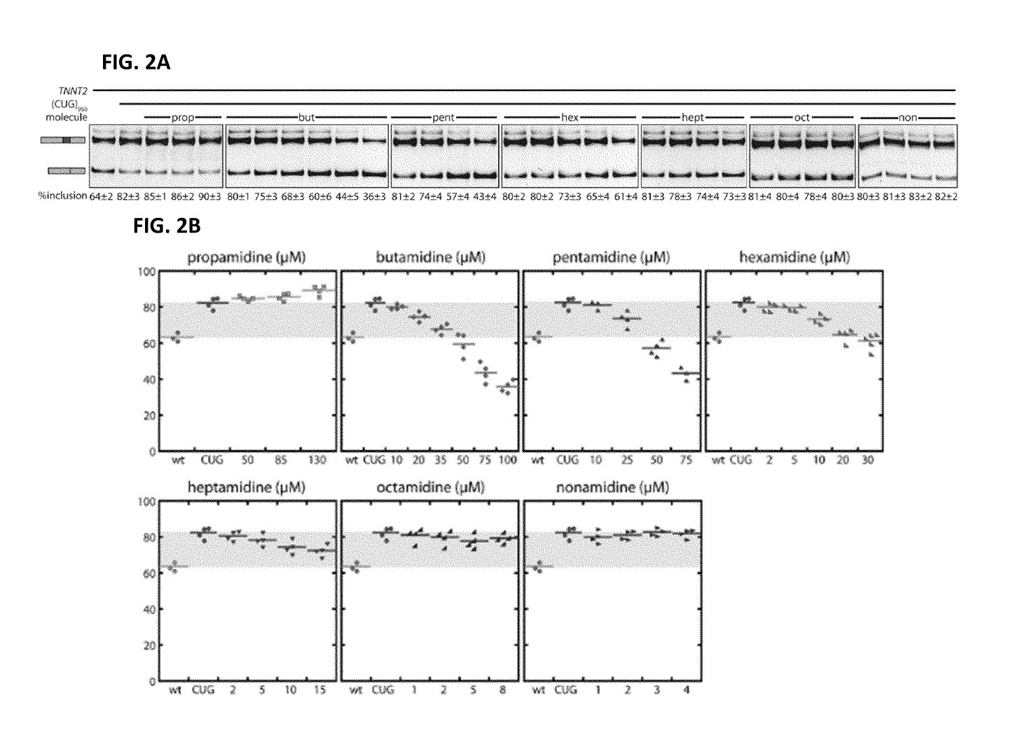

The ability of the linker analogues to rescue an exon that is positively regulated by MBNL proteins was also tested: exon 11 of the INSR gene. When wild type HeLa cells expressed the INSR reporter minigene, exon 11 inclusion was 71±1%. When 960 CUG repeats were expressed along with the INSR reporter, exon 11 inclusion dropped to 48±7% (FIG. 2C). All linker analogues were able to partially or fully rescue the mis-splicing of exon 11 of INSR when CUG repeats were expressed (FIG. 2D). Propamidine rescued mis-splicing with an EC₅₀ of 64±10 μM. Butamidine and pentamidine were similar with EC₅₀ values of 34±7 μM and 31±2 μM, respectively. Hexamidine also rescued INSR mis-splicing (EC50=15±1 μM), as did heptamidine (EC50=9±1 μM). Unlike with the TNNT2 minigene, both octamidine and nonamidine were able to rescue INSR mis-splicing with EC₅₀ values of 7±1 μM and 6±1 μM. As observed in the competitive EMSA, longer linker length leads to an increased in efficacy, particularly with respect to INSR (FIG. 3).

### Example 2

### Effect of Heptamidine In Vivo

### Methods

Heptamidine Treatment of Mice.

Homozygous HSALR transgenic mice in line 20b (FVB inbred background) were previously described (Mankodi et al., Science 289″1769-1773, 2000). Gender-matched mice of 10-14 weeks of age were treated with heptamidine at the indicated dose by daily intraperitoneal injection for 7 days. Control mice received 5% glucose injections. Mice were sacrificed 1 d after the final injection and vastus (quadriceps) muscle was obtained for splicing analysis. Mice in the withdrawal (WD) group were treated with 30 mg/kg heptamidine for 7 days and then left untreated for 10 days before sacrifice. Mice were sacrificed 1 day after final treatment and vastus muscle was obtained for splicing analysis. RNA was isolated, reverse transcribed, and amplified by PCR, and analyzed on agarose gels using a fluorimager as previously described (Warf et al., Proc. Natl. Acad. Sci. USA 106:18551-18556, 2009).

Electromyography:

Electromyography was performed under general anesthesia as described previously (Kanadia, Science 302:1978-1980, 2003). Briefly, at least 10 needle insertions were performed in vastus muscle and myotonic discharges were graded on a four point scale: 0, no myotonia; 1, occasional myotonic discharge in less than 50% of needle insertions; 2, myotonic discharge with more than 50% of insertions; and 3, myotonic discharge with nearly all insertions.

### Results

Heptamidine Rescues Mis-Splicing in a DM1 Mouse Model and Reduces Myotonia Symptoms:

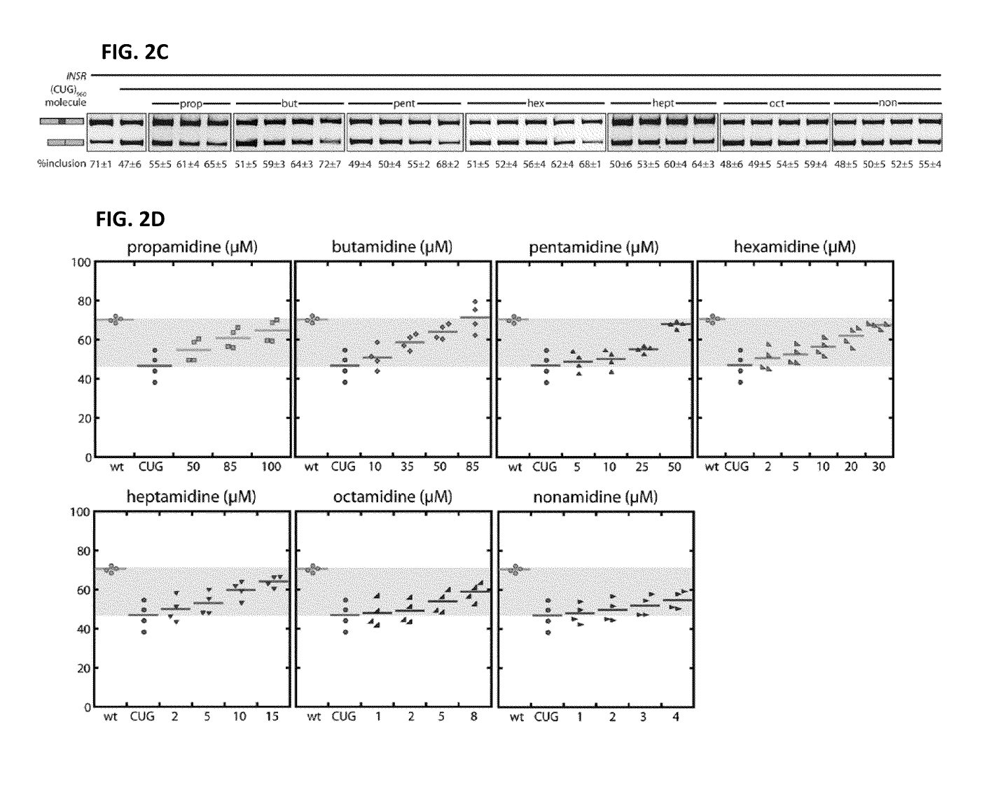

Because heptamidine disrupted the MBNL/(CUG)4 complex with the lowest IC₅₀ value and rescued mis-splicing in HeLa cells while retaining water solubility, it was tested in the HSA^{LR} transgenic DM1 mouse model. The HSA^{LR} DM1 mouse model expresses 220 CUG repeats under the skeletal promoter (Mankodi et al., Science 289″1769-1773, 2000). Two different endogenous pre-mRNAs were observed: Clcn1, the mis-splicing of which has been shown to cause myotonia (Mankodi et al., Mol. Cell. 10:35-44, 2002), and Atp2a1 (also called Serca1), which is a robust marker of mis-splicing in DM (Kimura, Hum. Mol. Genet. 14:2189-2200, 2005). MBNL proteins have been shown to promote exclusion of exon 7a of the Clcn1 gene. Wild type adult mice include exon 7a of the Clcn1 pre-mRNA at a level of 4±1% while HSA^{LR} mice include exon 7a at steady state levels of 47±1% (FIG. 3, note that exon 7a inclusion isoforms are subject to nonsense mediated decay). Treatment with heptamidine caused a dose-dependent reduction of exon 7a inclusion in HSALR mice, returning to wild type levels (6±1%) at the dose of 20 mg/kg heptamidine (FIG. 4).

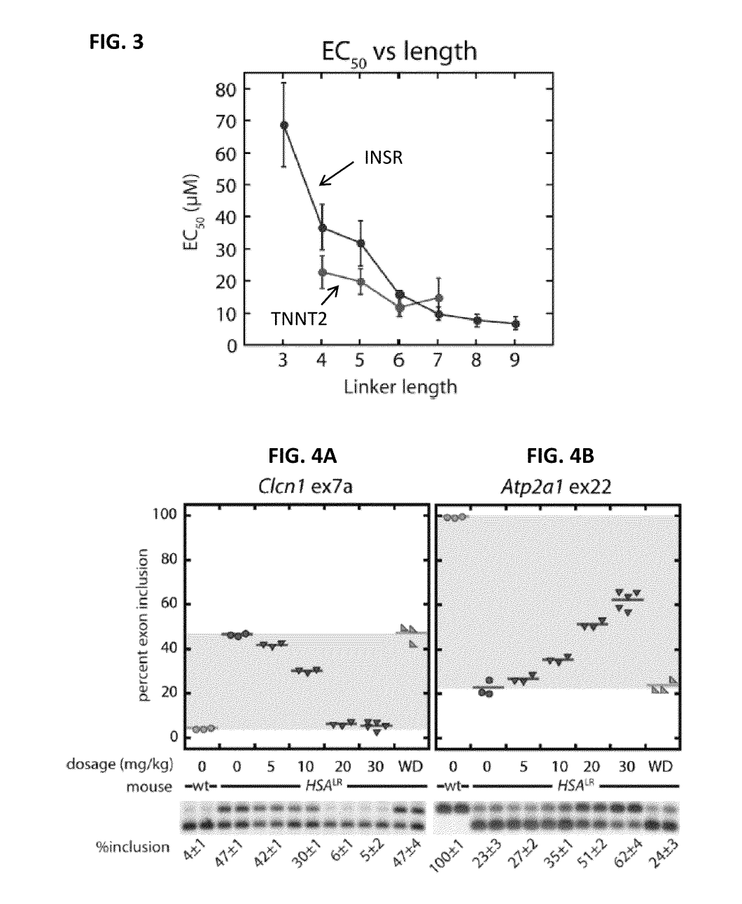

The MBNL regulated exon of Atp2a1 (exon 22) is included in the presence of MBNL proteins (positively regulated). Wild type adult mice included this exon 100±1% of the time (FIG. 4). When MBNL proteins are sequestered by the 220 CUG repeats present in HSALR mice, exon 22 inclusion drops to 23±3%. Although full rescue with heptamidine could not be reached, under a treatment regimen of 30 mg/kg/day for 7 days, exon 22 inclusion levels returned to 62±4% (FIG. 4). Additionally, after being treated with 30 mg/kg heptamidine for 7 d, mice went untreated for 10 days, and splicing of Clcn1 and Atp2a1 was examined. In both mRNAs, exon inclusion levels returned to control HSALA levels (FIG. 4). In addition to splicing defects, HSALR mice exhibit myotonia, manifested by runs of repetitive action potentials. The severity of myotonia was graded by insertion of extracellular recording electrodes into muscle tissue (electromyography) under general anesthesia. In glucose-treated controls, grade 3 myotonia was observed in vastus muscle, which indicates abundant repetitive discharges with nearly all electrode insertions. When treated with 20 or 30 mg/kg heptamidine, the myotonia was reduced from grade 3 to grade 1 (occasional myotonic discharge) or grade 0 (no myotonia) (FIG. 5). These results are consistent with Clcn1 splicing rescue observed under high heptamidine dosages, and suggest that by correcting DM1 mis-splicing events, myotonia symptoms can be alleviated.

### Example 3

### Activity of Pentamidine Prodrugs In Vitro

Prodrugs of pentamidine as shown below were synthesized.

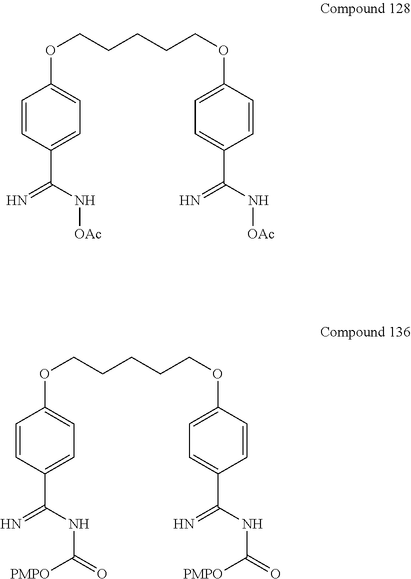

All compounds were prepared using analogous methods to those described by Tidwell et al. (J. Med. Chem. 33:1252-1257, 1990) and Clement et al. (Chem Med Chem 1:1260-1267, 2006).

### 1,3-bis(4-cyanophenoxy)pentane

Cyanophenol (5.0 g, 42.0 mmol) was dissolved in dioxane (50 mL) and potassium carbonate (12.2 g, 88.2 mmol) was added in a large portion, followed by 1,5-dibromopentane (2.86 mL, 21.0 mmol). The mixture was heated at reflux for 5 days, then concentrated with a rotary evaporator. The solid obtained was partitioned between methylene chloride (200 mL) and water (20 mL). The organic layer was washed additionally with water (20 mL) then dried with brine and sodium sulfate and concentrated to give white solid. This was recrystallized from dichloromethane gave the desired product in good purity, white crystals (9.64 g, 75%). ¹H NMR (300 MHz, CDCl₃) δ 1.64 (m, 2H), 1.89 (m, 4H), 4.03 (t, J=6.0 Hz, 4H), 6.92 (d, J=9.0 Hz, 4H), 7.57 (d, J=9.0 Hz, 4H).

### 1,5-bis(4-acetoxyamidinophenoxy)pentane (diacetyldiamidoximeester) (compound 128)

1,3-bis(4-chanophenoxy)pentane (250 mg, 0.82 mmol) was added to a 60° C. solution consisting of abs. methanol (10 mL), hydroxylamine hydrochloride (445 mg, 6.56 mmol), and triethylamine (0.82 mL, 6.56 mmol) that was obtained after heating and stirring the mixture at 60° C. for 30 min. Heating continued overnight, then the solution was poured into water (20 mL) and stored at 0° C. for 1 hour. The precipitate was collected on filter paper and rinsed with water (10 mL) and methylene chloride (20 mL) to give a tacky cake. The cake was mixed with acetic anhydride (5 mL) and stirred vigorously for 30 min. The mixture was diluted with ice cold water (20 mL) and stored at 0° C. for 1 h then the precipitate was collected on filter paper. The white solid obtained was recrystallized from acetonitrile to give the desired product in good purity, white powder (120 mg, 32%).

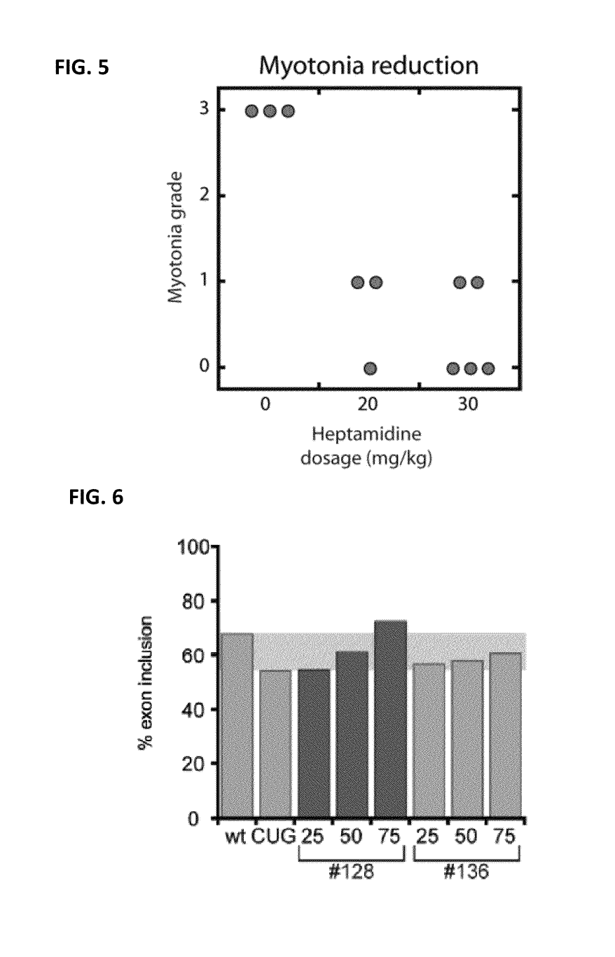

The pentamidine prodrug compounds were tested in the in vitro HeLa cell splicing assay for INSR splicing, as described in Example 2. These compounds, particularly compound 128, showed activity in the splicing assay (FIG. 6), while exhibiting decreased cell toxicity (HeLa cell death). The acetoxy-amidine prodrugs were less toxic than their amidine counterparts. For example, the acetoxy-amidine derivative of pentamidine was less toxic than pentamidine and the acetoxy-amdine derivative of heptamidine was less toxic than heptamidine.

### Example 4

### Identifying the Pharmacophore of Diamidines

Diamidine derivatives were synthesized to determine the molecular components that participate in breaking up MBNL/CUG repeat complexes. Aryl amidines with only alkyl phenolic chains, alkyl phenyl chains, or alkyl chains, and alkyl amidines were synthesized. The derivatives were synthesized from different combinations of commercially available phenolic benzonitriles and alkyl halides. Phenolates were generated under basic conditions and used to displace alkyl halides, yielding ethers. The nitrile groups are transformed via their Pinner salts with anhydrous HCl (g) and alcohol, followed by ammonia. The resulting aryl amidines were purified with reverse phase chromatography and recrystallized as the HCl salts.

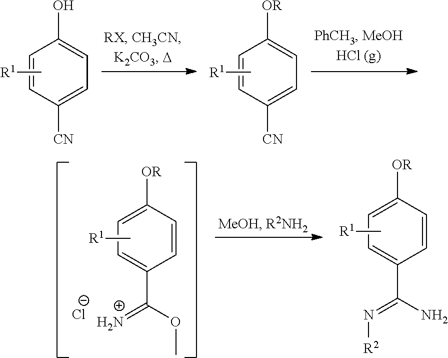

The diamidine derivatives were tested in vitro for their ability to effective at disrupting the MBNL1/(CUG)₄ complex (as described in Example 1). Removal of one amidine group or any portion of the diamidine molecule dramatically increased the IC₅₀ compared to the diamidine (Table 2; compare to Table 1).

TABLE 2
IC₅₀ of diamidine derivatives

| Formula | n = | IC50 (μM) |
|---|---|---|
| 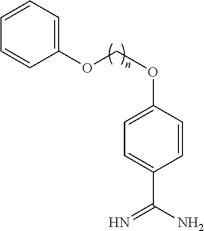 | 3 4 5 6 7 | 281 209 199 236 253 |
| 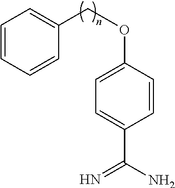 | 2 3 4 | 414 169 212 |
| 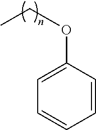 | 1 2 3 4 7 | >800 >800 >800 770 509 |

### Example 5

### Synthesis and Testing of Diamidine Prodrugs

Diamidine derivatives and prodrugs can be synthesized according to the methods provided herein (for example in Examples 3 and 4) and methods available to one of skill in the art. The compounds are tested in a cell model of DM for their ability to rescue splicing defects in an in vitro model of DM, as described in Example 1 and Warf et al. (Proc. Natl. Acad. Sci. USA 106:18551-18556, 2009). Compounds that exhibit rescue of splicing defects in a cell model (e.g. an IC₅₀ of about 200 μM or less) are selected for additional testing in an in vivo model of DM or other toxic RNA disease, as described in Example 2 and Warf et al. (Proc. Natl. Acad. Sci. USA 106:18551-18556, 2009). Compounds that achieve at least partial phenotypic rescue in an in vivo model are selected for further testing, for example in a Phase I or II clinical trial.

### Example 6

### Methods of Treating Myotonic Dystrophy

This example describes methods that can be used to treat DM in a subject. One skilled in the art will appreciate that, based on the teachings provided herein, methods that deviate from these specific methods can also be used to successfully treat DM.

In an example, a subject who has been diagnosed with DM1 or DM2 is identified. Following subject selection, an effective amount of a composition including an aryl diamidine or derivative or prodrug thereof is administered to the subject. The amount of the compound administered to prevent, reduce, inhibit, and/or treat DM depends on the subject being treated, the particular disorder, the severity of the disorder, and the manner of administration of the composition. Ideally, an effective amount of the compound is an amount sufficient to prevent, reduce, and/or inhibit, and/or treat the condition in the subject without causing a substantial cytotoxic effect in the subject.

In one specific example, the composition is administered intravenously or orally. For example, a composition including about 0.5-25 mg/kg of a disclosed diamidine derivative or prodrug is administered daily for at least 1 week. Subjects may be administered increasing doses of the composition, for example, if unacceptable side effects do not occur (“step-up” protocol), or different subjects may be administered different doses of the composition to identify a suitable dosage for treatment.

A reduction in the clinical symptoms associated with DM, for example, decreased muscle weakness, myotonia, or other pathological features (such as rows of internal nuclei, ring fibers, sarcoplasmic masses, type I fiber atrophy, and increased number of intrafusal muscle fibers in muscle biopsy) indicates the effectiveness of the treatment.

In view of the many possible embodiments to which the principles of the disclosure may be applied, it should be recognized that the illustrated embodiments are only examples and should not be taken as limiting the scope of the invention. Rather, the scope of the invention is defined by the following claims. We therefore claim as our invention all that comes within the scope and spirit of these claims.

We claim:

1. A compound comprising the structure: 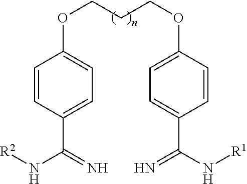
wherein n is 1 to 7, and R¹ and R² are independently selected from H, OR³, C(O)OR⁴, and SR⁵, wherein:
R³ is selected from H, C(O)(CH₂)ₘCH₃, C(O)C(CH₃)₃, (CH₂)ₘOC(O)C(CH₃)₃, SO₂Me, SO₂Tol, and C(O)CH(i-Pr)NH₂, wherein m is 0-10;
R⁴ is selected from (CH₂)ₘCH₃, (CH₂)ₘCX₃, (CH₂)ₘPh, (CH₂)ₘPh-X, (CH₂)ₘPh-Y, and (CH₂)ₘOC(O)C(CH₃)₃, wherein X is independently Cl, F, I, or Br, wherein Y is OR⁶, wherein R⁶ is (CH₂)ₘCH₃, and wherein m is 0-10; and
R⁵ is selected from (CH₂)ₘCH₃, C(CH₃)₃, Ph, and (CH₂)ₘC(O)O(CH₂)ₘCH₃, wherein each m is independently 0-10.

2. The compound of claim 1, wherein R¹ and R² are independently OR³ and R³ is selected from H, C(O)CH₃, C(O)C(CH₃)₃, CH₂OC(O)C(CH₃)₃, SO₂Me, SO₂Tol, and C(O)CH(i-Pr)NH₂.

3. The compound of claim 1, wherein R¹ and R² are independently C(O)OR⁴ and R⁴ is selected from CH₃, CH₂CCl₃, Ph, CH₂Ph, Ph-F, CH₂Ph-F, PhOCH₃, CH₂Ph)CH₃, and CH₂OC(O)C(CH₃)₃.

4. The compound of claim 1, wherein R¹ and R² are independently SR⁵ and R⁵ is selected from (CH₂)₂CH₃, C(CH₃)₃, Ph, and (CH₂)₂C(O)OCH₂CH₃

5. The compound of claim 1, wherein n is 3.

6. The compound of claim 1, wherein n is 5.

7. A composition comprising the compound of claim 1 and a pharmaceutically acceptable carrier.

8. A method of treating myotonic dystrophy in a subject, comprising administering to the subject an effective amount of the compound of claim 1.

9. A compound comprising the structure: 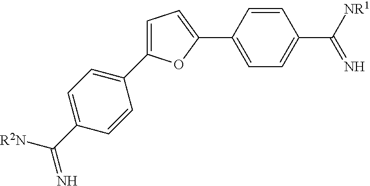
wherein R¹ and R² are independently selected from H, OR³, C(O)OR⁴, and SR⁵, wherein:
R³ is selected from H, C(O)(CH₂)ₘCH₃, C(O)C(CH₃)₃, (CH₂)ₘOC(O)C(CH₃)₃, SO₂Me, SO₂Tol, and C(O)CH(i-Pr)NH₂, wherein m is 0-10;
R⁴ is selected from (CH₂)ₘCH₃, (CH₂)ₘCX₃, (CH₂)ₘPh, (CH₂)ₘPh-X, (CH₂)ₘPh-Y, and (CH₂)ₘOC(O)C(CH₃)₃, wherein X is independently Cl, F, I, or Br, wherein Y is OR⁶, wherein R⁶ is (CH₂)ₘCH₃, and wherein m is 0-10; and
R⁵ is selected from (CH₂)ₘCH₃, C(CH₃)₃, Ph, and (CH₂)ₘC(O)O(CH₂)ₘCH₃, wherein each m is independently 0-10.

10. A composition comprising the compound of claim 9 and a pharmaceutically acceptable carrier.

11. A method of treating myotonic dystrophy in a subject, comprising administering to the subject an effective amount of the compound of claim 9.

12. A compound comprising the structure: 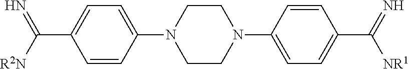
wherein R¹ and R² are independently selected from H, OR³, C(O)OR⁴, and SR⁵, wherein:
R³ is selected from H, C(O)(CH₂)ₘCH₃, C(O)C(CH₃)₃, (CH₂)ₘOC(O)C(CH₃)₃, SO₂Me, SO₂Tol, and C(O)CH(i-Pr)NH₂, wherein m is 0-10;
R⁴ is selected from (CH₂)ₘCH₃, (CH₂)ₘCX₃, (CH₂)ₘPh, (CH₂)ₘPh-X, (CH₂)ₘPh-Y, and (CH₂)ₘOC(O)C(CH₃)₃, wherein X is independently Cl, F, I, or Br, wherein Y is OR⁶, wherein R⁶ is (CH₂)ₘCH₃, and wherein m is 0-10; and
R⁵ is selected from (CH₂)ₘCH₃, C(CH₃)₃, Ph, and (CH₂)ₘC(O)O(CH₂)ₘCH₃, wherein each m is independently 0-10.

13. A composition comprising the compound of claim 12 and a pharmaceutically acceptable carrier.

14. A method of treating myotonic dystrophy in a subject, comprising administering to the subject an effective amount of the compound of claim 12.

15. A compound comprising the structure: 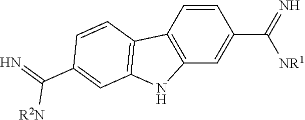
wherein R¹ and R² are independently selected from H, OR³, C(O)OR⁴, and SR⁵, wherein:
R³ is selected from H, C(O)(CH₂)ₘCH₃, C(O)C(CH₃)₃, (CH₂)ₘOC(O)C(CH₃)₃, SO₂Me, SO₂Tol, and C(O)CH(i-Pr)NH₂, wherein m is 0-10;
R⁴ is selected from (CH₂)ₘCH₃, (CH₂)ₘCX₃, (CH₂)ₘPh, (CH₂)ₘPh-X, (CH₂)ₘPh-Y, and (CH₂)ₘOC(O)C(CH₃)₃, wherein X is independently Cl, F, I, or Br, wherein Y is OR⁶, wherein R⁶ is (CH₂)ₘCH₃, and wherein m is 0-10; and
R⁵ is selected from (CH₂)ₘCH₃, C(CH₃)₃, Ph, and (CH₂)ₘC(O)O(CH₂)ₘCH₃, wherein each m is independently 0-10.

16. A composition comprising the compound of claim 15 and a pharmaceutically acceptable carrier.

17. A method of treating myotonic dystrophy in a subject, comprising administering to the subject an effective amount of the compound of claim 15.

18. A compound comprising the structure: 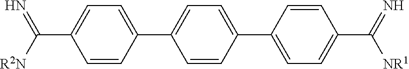
wherein R¹ and R² are independently selected from H, OR³, C(O)OR⁴, and SR⁵, wherein:
R³ is selected from H, C(O)(CH₂)ₘCH₃, C(O)C(CH₃)₃, (CH₂)ₘOC(O)C(CH₃)₃, SO₂Me, SO₂Tol, and C(O)CH(i-Pr)NH₂, wherein m is 0-10;
R⁴ is selected from (CH₂)ₘCH₃, (CH₂)ₘCX₃, (CH₂)ₘPh, (CH₂)ₘPh-X, (CH₂)ₘPh-Y, and (CH₂)ₘOC(O)C(CH₃)₃, wherein X is independently Cl, F, I, or Br, wherein Y is OR⁶, wherein R⁶ is (CH₂)ₘCH₃, and wherein m is 0-10; and
R⁵ is selected from (CH₂)ₘCH₃, C(CH₃)₃, Ph, and (CH₂)ₘC(O)O(CH₂)ₘCH₃, wherein each m is independently 0-10.

19. A composition comprising the compound of claim 18 and a pharmaceutically acceptable carrier.

20. A method of treating myotonic dystrophy in a subject, comprising administering to the subject an effective amount of the compound of claim 18.


SEQUENCE LISTING (7 sequences)

SEQ ID NO: 1 - RNA, 17 nt, Artificial Sequence
  Synthetic oligonucleotide construct
gcugcuguucgcugcug

SEQ ID NO: 2 - DNA, 28 bp, Artificial Sequence
  Synthetic oligonucleotide primer - TNNT2 RT primer
agcatttaggtgacactatagaataggg

SEQ ID NO: 3 - DNA, 26 bp, Artificial Sequence
  Synthetic oligonucleotide primer - TNNT2 forward
gttcacaaccatctaaagcaagatgs

SEQ ID NO: 4 - DNA, 19 bp, Artificial Sequence
  Synthetic oligonucleotide primer - TNNT2 reverse
gttgcatggctggtgcagg

SEQ ID NO: 5 - DNA, 21 bp, Artificial Sequence
  Synthetic oligonucleotide primer - INSR RT primer
gctgcaataaacaagttctgc

SEQ ID NO: 6 - DNA, 32 bp, Artificial Sequence
  Synthetic oligonucleotide primer - INSR forward
cgaattcgaatgctgctcctgtccaaagacag

SEQ ID NO: 7 - DNA, 20 bp, Artificial Sequence
  Synthetic oligonucleotide primer - INSR reverse
tcgtgggcacgctggtcgag